(19) United States
(12) Patent Application Publication
(10) Pub. No.: US 2021/0392923 A1
(43) Pub. Date: Dec. 23, 2021

(54) BLEND FORMULATION COMPRISING SILICATE AND MICROBIAL AND / OR PLANT CELLS COMPRISING A POLYUNSATURATED FATTY ACID HAVING AT LEAST 20 CARBON ATOMS (LC-PUFA)

(71) Applicants: DSM IP ASSETS B.V., Heerlen (NL); EVONIK OPERATIONS GMBH, Essen (DE)

(72) Inventor: Dirk Luc DE VRIENDT, Kaiseraugst (CH)

(21) Appl. No.: 17/411,029

(22) Filed: Aug. 24, 2021

Related U.S. Application Data
(63) Continuation of application No. 16/469,286, filed on Jun. 13, 2019, now abandoned.

(30) Foreign Application Priority Data
Dec. 15, 2016 (EP) 16204404.4

(51) Int. Cl. A23K 20/28; A23K 40/10; A23K 10/30; A23L 33/115; A23L 29/00; A23D 9/007; A23D 9/05; C12P 7/64

(52) U.S. Cl. CPC A23K 20/28; A23K 40/10; A23K 10/30; A23L 33/115; A23K 50/10; A23D 9/007; A23D 9/05; C12P 7/6427; A23L 29/015

(57) ABSTRACT

The present invention relates to a blend formulation (I) comprising
(i) at least 50 weight-% (wt-%), based on the total weight of the blend formulation, of a LC-PUFA-containing composition
(ii) 10-50 wt-%, based on the total weight of silcate.

This application is a continuation of commonly owned copending U.S. application Ser. No. 16/469,286, filed Jun. 13, 2019 (now abandoned), which is the U.S. national phase of International Application No. PCT/EP2017/082755 filed Dec. 14, 2017, which designated the U.S. and claims priority to EP Patent Application No. 16204404.4, filed Dec. 15, 2016, the entire contents of each of which are hereby incorporated by reference.

The present invention relates to a blend formulation comprising cells and a polyunsaturated fatty acid having at least 20 carbon atoms (LC-PUFA).

LC-PUFAs can be produced by micro-organisms in a fermentation process. LC-PUFAs can also be produced in plants. The microorganisms or plant parts containing the LC-PUFA can then be pre-treated after which LC-FUFA or oil containing the LC-PUFA can be isolated.

For instance, WO 2006/085672, describes a process wherein an LC-PUFA is isolated from a microbial biomass. Wet cells are dried in a two-stage drying process. Drying temperatures of 120° C. and higher are used, and dried cells having a moisture content of 1-2 wt. % are obtained. It is possible to isolate the LC-PUFA and/or oil containing the LC-PUFA from the LC-PUFA-containing composition immediately after its production. However, in practice the LC-PUFA-containing composition is often stored and/or transported before further use such as isolation of the LC-PUFA and/or oil containing the LC-PUFA. All these various compositions are called LC-PUFA-containing compositions.

It is now found that LC-PUFA-containing compositions are susceptible to self-heating. Viz. during storage, the temperature can increase spontaneously, ultimately resulting in unexpected explosions and fires. It is further found that this susceptibility increases with increasing LC-PUFA content, and with increasing number of double bonds of the LC-PU FAs.

It is an object of the invention to provide a composition comprising (i) LC-PUFA and (ii) cells, which composition is safer.

In particular, there is an ongoing need to develop a product form, which overcomes the above mentioned problem, but has still a good flowability and can easily be admixed with other components commonly used in feed products for ruminants.

Surprisingly, it has been found that a LC-PUFA-containing composition as described above is effectively retained and safe in silicate as for example in Sepiolite (magnesium silicate) under conventional storage conditions.

Thus in a first embodiment the present invention relates to a blend formulation (I) comprising
(i) at least 50 weight-% (wt-%), based on the total weight of the blend formulation, of a LC-PUFA-containing composition
(ii) 10-50 wt-%, based on the total weight of silicate.

The blend formulations according to the present invention are powders, which depending on the process of production as well as the storage conditions, may furthermore contain small amounts of customary additives commonly used in the preparation of blends and premixes for feed application. Therefore a further embodiment of the present invention relates to formulations according to the present invention, wherein 0 to 5 wt-%, based on the total weight of the formulation, of an additive is present.

It is clear that in all embodiments of the present invention the addition of all the wt-% always add up to 100. However, it cannot be excluded that small amount of impurities may be pre-sent such as e.g. in amounts of less than 5 wt. %, preferably less than 3 wt.-% which are introduced via the respective raw materials or processes used.

A silicate is a compound containing an anionic silicon compound. The great majority of the silicates are oxides, but hexafluorosilicates and other anions are also included. Silicates constitute the majority of Earth's crust, as well as the other terrestrial planets, rocky moons, and asteroids. Sand, Portland cement, and thousands of minerals are examples of silicates. Silicate compounds, including the minerals, consist of silicate anions whose charge is balanced by various cations. Myriad silicate anions can exist, and each can form compounds with many different cations. Hence this class of compounds is very large. Both minerals and synthetic materials fit in this class.

The term sepiolite as used herein refers to a soft white clay mineral consisting of hydrous magnesium silicate, a typical chemical formula for which is Mg4Si6O15(OH)2*6H2O and which can be present in fibrous, fine-particulate, and solid forms. Commercially available sepiolite grades suitable for the purpose of the present invention encompass EXAL H 562, 1530 and 3060 which are commercially available from Tolsa (Spain).

Preferably the sepiolite according to the present invention has an average particle size D(v, 0.5) selected in the range of 100 to 1500pm, more preferably in the range of 200 to 1250 μm and most preferably in the range of 200 to 1250 μm.

In a more advantageous embodiment, the sepiolite according to the present invention has a D(v, 0.5) selected in the range of 200 to 1250 μm, and in particular also a D(v, 0.1) selected in the range of 100 to 800 μm and a D(v, 0.9) selected in the range of 300 to 1750 μm.

The particle sizes as given herein are measured by a Malvern Master Sizer 2000 following the recommendations outlined in IS013320-1 for particle size analysis via laser diffraction methods (laser diffraction light scattering). During this laser diffraction measurement, parti-cles are passed through a focused laser beam. The particles scatter light at an angle that is inversely proportional to their size. The angular intensity of the scattered light is then meas-ured by a series of photosensitive detectors. The map of scattering intensity versus angle is the primary source of information used to calculate the particle size. For the measurement of sepiolite, a dry powder feeder (Malvern Scirocco) was used.

In a preferred embodiment, the LC-PUFA-containing composition has an oil content of at least 10 wt. %, for instance at least 20 wt. %, for instance at least 30 wt. %, for instance at least 40 wt. %. The oil content may be below 70 wt. %, for instance below 60 wt. %. The oil content may be determined by methods known to the skilled person. A suitable method for determining the oil content of the composition as used herein is by using a Soxhlet extraction using n-hexane as the solvent, wherein the composition subjected to the extraction has a moisture content <15 wt. % and wherein the composition and cells are comminuted (to ensure that all oil is released from the cells and can dissolve into the solvent). As used herein, the oil content is calculated on a dry basis, i.e. on the basis of the total dry weight of the composition (including dry matter and oil, but excluding moisture).

In a preferred embodiment, the LC-PUFA-containing composition has an oil content as defined above, wherein the composition of the oil is as in the preferred embodiments described below.

In a preferred embodiment, LC-PUFA-containing composition comprises an oil which comprises at least 10 wt. %, for instance at least 20 wt. %, for instance at least 30 wt. %, for instance at least 40 wt. % of PUFAs with at least 3 double bonds with respect to the total fatty acids in the oil, for instance below 80 wt. %, for instance below 70 wt. %, for instance below 60 wt. % of PUFAs with at least 3 double bonds with respect to the total fatty acids in the oil. As used herein, the wt. % of PUFAs with at least 3 double bonds refers to the sum of all PUFAs with at least 3 double bonds.

In a preferred embodiment, the LC-PUFA-containing composition comprises an oil which comprises at least 10 wt. %, for instance at least 20 wt. %, for instance at least 30 wt. %, for instance at least 40 wt. % of arachidonic acid (ARA) with respect to the total fatty acids in the oil, for instance below 80 wt. %, for instance below 70 wt. %, for instance below 60 wt. % ARA with respect to the total fatty acids in the oil.

In a preferred embodiment, the LC-PUFA-containing composition comprises an oil which comprises at least 10 wt. %, for instance at least 20 wt. %, for instance at least 30 wt. %, for instance at least 40 wt. % of docosahexaenoic acid (DHA) with respect to the total fatty acids in the oil, for instance below 80 wt. %, for instance below 70 wt. %, for instance below 60 wt. % DHA with respect to the total fatty acids in the oil.

A suitable method for determining the composition of the oil as used herein is to extract the oil from the composition using the Soxhlet extraction using n-hexane as described hereinabove, and to determine the fatty acid composition of the extracted oil.

It is preferred to select lower drying temperatures and shorter residence times in the dryer if the oil content and/or number of double bonds is relatively high.

As used herein, the following abbreviations are used throughout the entire application:
PUFA refers to a polyunsaturated fatty acid
LC-PUFA (long chain polyunsaturated fatty acid) refers to a PUFA having at least 20 carbon atoms
HUFA (highly unsaturated fatty acid) refers to a PUFA having at least three double bonds
LC-HUFA (long chain highly unsaturated fatty acid) refers to a polyunsaturated fatty acid having at least 20 carbon atoms and at least three double bonds.

The invention is not limited to a specific LC-PUFA. In an embodiment of the invention, the LC-PUFA has at least three double bonds. In a further embodiment of the invention, the LC-PUFA has at least four double bonds. The benefits of the invention are even more pronounced for LC-PUFAs having an increasing number of double bonds, as the susceptibility to self heating increases with increasing number of double bonds.
The LC-PUFA may be an ω-3 LC-PUFA or an ω-6 LC-PUFA
LC-PUFAs include for instance:
dihomo-y-linolenic acid (DGLA, 20:3 ω-6)
arachidonic acid (ARA, 20:4 ω-6)
eicosapentaenoic acid (EPA, 20:5 ω-3)
docosapentaenoic acid (DPA, 22:5 ω-3, or DPA 22:5, ω-6),
docosahexaenoic acid (DHA: 22:6 ω-3)

Preferred LC-PUFAs include arachidonic acid (ARA), eicosapentaenoic acid (EPA) and docosahexaenoic acid (DHA). In particular DHA is preferred.

The LC-PUFA-containing composition according to the invention comprises cells. The cells may be any cells containing and/or having produced the LC-PUFA.

In an embodiment of the invention, the cells are microbial cells (microorganisms). Examples of microbial cells are yeast cells, bacterial cells, fungal cells, and algal cells. Fungi are preferred, preferably of the order Mucorales. Example are Mortierella, Phycomyces, Blakeslea, Aspergillus, Thraustochytrium, Pythium or Entomophthora. The preferred source of arachidonic acid (ARA) is from Mortierella alpina. Algae can be dinoflagellate and/or include Porphyridium, Nitszchia, or Crypthecodinium (e.g. Crypthecodinium cohnii). Yeasts include those of the genus Pichia or Saccharomyces, such as Pichia ciferii. Bacteria can be of the genus Propionibacterium.

In an embodiment of the invention, the LC-PUFA-containing composition comprises a fungus of the genus Mortierella, preferably of the species Mortierella alpina, wherein preferably the LC-PUFA is ARA or DGLA.

In an embodiment of the invention the LC-PUFA-containing composition comprises a fungus of the order Thraustochytriales, for instance from the genus Thraustochytrium or Schizochytrium, and wherein preferably the LC-PUFA is DHA and/or EPA.

In an embodiment of the invention, the LC-PUFA-containing composition comprises an algae of the genus Crypthecodinium, preferably of the species Crypthecodinium cohnii, wherein preferably the LC-PUFA is DHA.

In another embodiment of the invention, the cells are plant cells. The cells may be plant cells of a transgenic plant.

Suitable plants and seeds are for instance described in WO 2005/083093, WO 2008/009600, and WO 2009/130291, the contents of which are hereby incorporated by reference. Other plants and seeds that can be used in the invention are for instance disclosed in WO 2008/100545, WO 2008/124806, WO 2008/124048, WO 2008/128240, WO 2004/071467, WO 2005/059130, the contents of which are hereby incorporated by reference. The seeds may be (transgene) soybeans or (transgene) canola seeds. The plant may be a (transgene) soybean plant or a (transgene) canola plant.

In a preferred embodiment, the plant is a (transgenic) plant of the family Brassicaceae, for instance the genera Brassica, Camelina, Melanosinapis, Sinapis, Arabidopsis, for example the genera and species Brassica alba, Brassica carinata, Brassica hirta, Brassica napus, Brassicaa rapa ssp., Sinapis arvensis, Brassica juncea, Brassica juncea var. juncea, Brassica juncea var. crispifolla, Brassica juncea var. foliosa, Brassica nigra, Brassica sinapioides, Camelina sativa, Melanosinapis communis, Brassica oleracea or Arabidopsis thaliana.

The LC-PUFA-containing composition may be any biomass comprising a LC-PUFA. Preferably, the composition is a (dried) composition obtained or obtainable by a drying process disclosed herein.

The LC-PUFA-containing composition may be a microbial biomass comprising a microorganism and a LC-PUFA. Preferred microorganisms and LC-PUFAs are mentioned hereinabove.

In a possible embodiment of the invention a composition comprising microorganisms (microbial cells) according to the invention is obtained in a process comprising heating (also referred to as pasteurization or sterilization) a fermentation broth comprising the microbial cells, dewatering the microbial cells, e.g. by filtration, and drying the microbial cells in a process described hereinabove. In a preferred embodiment, the dewatered microbial cells are granulated prior to drying, preferably by extrusion. Preferably granulation, e.g. extrusion is performed at a temperature below 25° C. A preferred process is described in WO 97/037032 which is herewith incorporated by reference.

In an embodiment of the invention, the LC-PUFA-containing composition comprises seeds comprising an LC-PUFA and/or the composition may be in the form of seeds. Preferably, the seeds are seeds of plants mentioned hereinabove.

Preferably, the seeds comprise at least 5 wt. %, preferably at least 10 wt. %, preferably at least 15 wt. %, preferably at least 20 wt. % of an LC-PUFA (for instance an LC-PUFA as described herein, with respect to the total fatty acids in the seeds.

Preferably, the seeds comprise at least 5 wt. %, preferably at least 10 wt. %, preferably at least 15 wt. %, preferably at least 20 wt. % of an □-6 LC-PUFA, with respect to the total fatty acids in the seeds.

Preferably, the seeds comprise at least 5 wt. %, preferably at least 10 wt. %, preferably at least 15 wt. %, preferably at least 20 wt. % of ARA, with respect to the total fatty acids in the seeds.

Preferably, the seeds comprise at least 5 wt. %, preferably at least 10 wt. %, preferably at least 15 wt. %, preferably at least 20 wt. % of an □-3 LC-PUFA, with respect to the total fatty acids in the seeds.

Preferably, the seeds comprise at least 5 wt. %, preferably at least 10 wt. %, preferably at least 15 wt. %, preferably at least 20 wt. % of DHA, with respect to the total fatty acids in the seeds.

Preferably, the seeds comprise less than 2 wt. % of erucic acid, preferably less than 1 wt. %, preferably less than 0.5 wt. % based on the total fatty acids in the seeds.

The powderous blend formulation according to the present invention comprises
(i) at least 50 weight-% (wt-%), based on the total weight of the blend formulation, of a LC-PUFA-containing composition and
(ii) 10 - 50 wt-%, based on the total weight of a silcate.

Preferred ratios of LC-PUFA-containing composition/Silicate powder are: 50/50; 60/40; 70/30; 80/20; 90/10.

The powderous blend formulation according to the present invention can additionally be coated with customary coatings in the art such as wax or fats. If present, such coating is generally applied in amounts of 5 to 50 wt.-% based on the total weight of the powderous form. Advantageously, the coating comprises at least one wax and/or at least one fat, which has a drop-ping point of from 30 to 85° C.

Particularly suitable fats to be used as coating in the context of the present invention include a wide group of compounds which are soluble in organic solvents and largely insoluble in water such as hydrogenated fats (or saturated fats) which are generally triesters of glycerol and fatty acids. Suitable fats can have natural or synthetic origin. It is possible to hydrogen-ate a (poly)unsaturated fat to obtain a hydrogenated (saturated) fat.

Preferred examples of waxes and fats to be used as coating according to the present inven-tion are glycerine monostearate, carnauba wax, candelilla wax, sugarcane wax, palmitic acid, stearic acid hydrogenated cottonseed oil, hydrogenated palm oil and hydrogenated rapeseed oil as well as mixtures thereof.

All the above disclosed blend formulations (I), (II), (Ill), (IV), (V) can be used as such or incorporated in feed products.

The blend formulation according to the invention may suitably be stored prior to further use and/or processing.

Advantageously, the blend formulation is stored at a temperature of below 10° C., preferably below 5° C., preferably below 0° C., preferably below minus 5° C., preferably below minus 10° C. There is no specific lower limit for the storage temperature. Generally, the composition is stored at a temperature of above minus 30° C.

If the blend formulation comprises seeds or is in the form of seeds, preferably the seeds have a moisture content of less than 15 wt. %, for instance less than 12 wt. %, for instance less than 10 wt. %, for instance less 9.5 wt. %, for instance above 6 wt. %, for instance above 7 wt. %, for instance above 8 wt. %. The moisture content may for instance be between 6 and 15 wt. %, for instance between 7 and 12 wt. %, for instance between 8 and 10 wt. %. The preferred moisture contents can be obtained by drying the seeds as described hereinabove.

The blend formulation may be stored for any suitable period. The composition may for instance be stored for at least 1 day, for instance at least 1 week, for instance at least 2 weeks, for instance at least 1 months, for instance at least 3 months. There is no specific upper limit for the storage period. The composition, may for instance be stored for less than 12 months, for instance less than 6 months.

Further preferred aspects, embodiments and features are disclosed in the claims Preferred features and characteristics of one embodiment and/or aspect of the invention are applicable to another embodiment mutatis mutandis. As used herein, the preferred features and characteristics of the LC-PUFA apply to the LC-PUFAs in all aspects and embodiments of the invention.

The invention is further disclosed with reference to the following examples without being limited thereto.

### EXAMPLES

### Example 1

Fermentation broth of Mortierella alpina, obtained after 8 days of fermentation was pasteurized at 70° C. for 1 hour. The pasteurized broth was filtered, resulting in a filter cake have a moisture content of 50 wt. %. The filter cake was crumbled and extruded at a temperature below 15° C. The extrudate (diameter 3 mm) was dried in a continuous fluid bed drier with three zones to a moisture content of 7%. In the first zone the bed temperature was 32° C. and the air temperature 50° C. (T_{dew point}=15° C.). 1^{st} zone: bed temperature 32° C., the air temperature 50° C. (T_{dew point}=15° C.): 45 minutes 2^{nd} zone: bed temperature 32° C., air temperature 35° C. (T_{dew point}=1° C.): 45 minutes 3^{rd} zone: bed temperature 15° C., air temperature 15° C. (T_{dew point}=1° C.): 30 minutes The oil content of the dried biomass was 39%. The ARA content was 46% with respect to the total fatty acids in the oil.

For the preparation of the blend formulation according to the invention, the silica compound is blended with the dried biomass in a separate mixing step.

### Example 2

Seeds containing 19% Arachidonic acid (with respect to total fatty acids) are obtained from transgenic Brassica plants that are transformed using the procedures described in WO2008009600.

The seeds have the following specifications (determined in accordance with the Official Grain Grading Guide, 2001 of the Canadian Grain Commission): distinctly green<2%, total damaged<5%.

The seeds, having a moisture content of 17 wt. %, are dried using a fluid bed drier. The bed temperature is 28° C. Conditioned air is used having a dew point of 10° C. The dried seeds have a moisture content of 8.5 wt. %. The oil content is 35 wt. %.

For the preparation of the blend formulation according to the invention, the silica compound is blended with the dried seed in a separate mixing step.

### Example 3 Silicate Powder for Use According to the Invention

Sepiolite: Particle size

| Sepiolite | D1 | D2 | D3 |
|---|---|---|---|
| Sepiolite type 1 | 170 μm | 367 μm | 412 μm |
| Sepiolite type 2 | 713 μm | 1038 μm | 1486 μm |
| Sepiolite type 3 | 424 μm | 631 μm | 950 μm |

1. A powderous blend formulation (I) comprising:
(i) microbial or plant cells,
(ii) at least 50 weight percent (wt. %), based on the total weight of the blend formulation, of a composition comprising a long chain polyunsaturated fatty acid (LC-PUFA-containing composition); and
(iii) 10-50 wt. %, based on the total weight of the blend formulation, of a silicate.

2. The powderous blend formulation according to claim 1, further comprising 0 to 10 wt. %, based on the total weight of the blend formulation, of water and/or an additive.

3. The powderous blend formulation according to claim 1, wherein the silicate consists of sepiolite.

4. The powderous blend formulation according to claim 3, wherein the sepiolite has an average particle size D(v, 0.5) in a range of 100 to 1500 μm.

5. The powderous blend formulation according to claim 1, wherein powder particles of the powderous blend formulation comprise a coating.

6. The powderous blend formulation according to claim 5, wherein the coating is selected from the group consisting of glycerine monostearate, carnauba wax, candelilla wax, sugarcane wax, palmitic acid, stearic acid hydrogenated cottonseed oil, hydrogenated palm oil, hydrogenated rapeseed oil and mixtures thereof.

7. The powderous blend formulation according to claim 1, wherein the LC-PUFA-containing composition has a moisture content of between 1 and 20 wt. %.

8. The powderous blend formulation according to claim 7, wherein the LC-PUFA-containing composition has an oil content of at least 10 wt. %.

9. The powderous blend formulation according to claim 8, wherein the LC-PUFA-containing composition comprises an oil which comprises at least 10 wt. % to below 80 wt. % of polyunsaturated fatty acids (PUFAs) with at least 3 double bonds with respect to total fatty acids in the oil.

10. The powderous blend formulation according to claim 7, wherein the long chain polyunsaturated acids (LC-PUFAs) in the LC-PUFA-containing composition have at least 3 double bonds.

11. The powderous blend formulation according to claim 1, wherein the cells comprise a microorganism of the order Thraustochytriales.

12. The powderous blend formulation according to claim 1, wherein the cells comprise a microorganism of the genus Thraustochytrium or Schizochytrium.

13. The powderous blend formulation according to claim 1, wherein the cells comprise docosahexaenoic acid (DHA) or eicosapentaenoic acid (EPA).

14. Feed products comprising the powderous blend formulation according to claim 1.
